(19) United States
(12) Patent Application Publication
(10) Pub. No.: US 2010/0055172 A1
(43) Pub. Date: Mar. 4, 2010

(54) ENCAPSULATED SOY EXTRACTS AND PROCESS FOR PREPARING SAME

(76) Inventor: Choi Yong Han, Kyunggi-Do (KR)

Correspondence Address: FOLEY & LARDNER LLP, 150 EAST GILMAN STREET, P.O. BOX 1497, MADISON, WI, 53701-1497, US

(21) Appl. No.: 12/444,418

(22) PCT Filed: Nov. 1, 2007
(86) PCT No.: PCT/US2007/083323

§ 371 Date: Apr. 6, 2009

Related U.S. Application Data
(60) Provisional application No. 60/864,217, filed on Nov. 3, 2006.

(51) Int. Cl. A61K 9/48; A61K 36/48

(52) U.S. Cl. 424/451; 424/757

(57) ABSTRACT

Soy extracts encapsulated with oligosaccharides and processes for making the encapsulated soy extracts. The processes generally comprise combining a soy extract solution and an oligosaccharide solution to encapsulate the soy extract with the oligosaccharide, precipitation of the encapsulated soy extract solution to form sediment, centrifugation, ion exchange to remove ionic components and drying. Soy extracts encapsulated in the process include isoflavone derivatives, such as daidzin, glycitin, genistin, daidzein, glycitein and genistein, which may be encapsulated with αr-cyclodextrin, β-cyclodextrin, γ-cyclodextrin or combinations of these oligosaccharides.

### CROSS REFERENCE TO RELATED APPLICATION

This application claims the benefit of U.S. Provisional Application No. 60/864,217 filed Nov. 3, 2006, assigned to the assignee of this application and incorporated by reference herein.

### BACKGROUND OF THE INVENTION

1. Field of the Invention

The invention pertains to soy extracts encapsulated with oligosaccharides. The invention also relates to processes for encapsulating soy extracts.

2. The Related Art

Soy is a food product that contains many nutritionally valuable constituents like isoflavones, saponins, phytosterols and essential amino acids. Soy extracts, such as isoflavone derivatives, when included in the human diet provide beneficial physiological activities, for example, estrogen action, antioxidant activity, and antimicrobial activity. Soy extracts, particularly isoflavones, are considered as possible inhibitors of certain types of cancers, such as breast cancer and prostate cancer. Isoflavone derivatives are reported to suppress decreased bone density in ovariectomized rats with a low calcium diet and, thus, isoflavone may be considered as effective in preventing or inhibiting osteoporosis. Soy extracts may also enhance immune function and are reported to lower cholesterol levels when incorporated into the human diet.

All parts and percentages set forth in this specification are on a weight by weight basis unless otherwise specified.

### SUMMARY OF THE INVENTION

The invention pertains to encapsulated soy extracts. The encapsulated soy extracts comprise one or more soy extracts and one or more oligosaccharides. The soy extracts are encapsulated with the oligosaccharides. The encapsulated soy extracts comprise little or no impurities, and have complete or nearly complete solubility in solvent, such as water.

The invention also pertains to processes for preparing the encapsulated soy extracts, or encapsulated derivates of the soy extract. The processes generally comprise preparing a soy extract solution and an oligosaccharide solution and then encapsulating the soy extract with the oligosaccharide with a means for encapsulation, such as a homomixer, homogenizer or stirrer. The process may further comprise steps of precipitation, centrifugation, ion exchange and drying such as in a spray drier.

### DESCRIPTION OF THE DRAWING

FIG. 1 is a flow diagram showing a process of an embodiment of the invention for preparing the encapsulated soy extracts.

### DETAILED DESCRIPTION OF THE INVENTION

The invention pertains to encapsulated soy extracts and processes for making the encapsulated soy extracts with minimal or no impurities. Soy extracts generally comprise isoflavones, soluble dietary fiber, sugar, fat, protein or the like. The primary impurities in soy extracts are protein and fat and the like which lead to insolubility of the soy extract in water. The term “primary impurities” as used herein shall refer to protein, fat and the like, and the term “impurities” shall include primary impurities and other materials that are not isoflavone and/or isoflavone derivatives. Raw soy extracts will comprise more than about 10% primary impurities. For example, the soy extracts prior to processing may comprise about 9% protein and about 3% fat. After the soy extract is encapsulated by the processes discussed herein the encapsulated soy extract will comprise less than about 1.0%, such as less than about 0.7%, primary impurities. The encapsulated soy extract may comprise from about 0.1% to about 1.0% primary impurities, such as from about 0.1% to about 0.7% primary impurities. The encapsulated soy extract may comprise about 0.1% to about 0.9% protein and/or about 0.1% to about 0.9% fat. The encapsulated soy extracts made by the processes described herein have complete or nearly complete solubility in water, such as about 99% to 100% in water based on a solution of about 1% and it is believed that this is the result, at least in part, from the low levels of impurities, particularly primary impurities, in the encapsulated product made by the processes described herein.

The preferred soy extract is isoflavone, particularly isoflavone derivatives, such as those selected from the group consisting of daidzin, glycitin, genistin, daidzein, glycitein, genistein and combinations thereof. Other soy extracts are within the scope of the invention. The soy extracts, i.e., isoflavone, such as the isoflavone derivatives, are encapsulated with one or more oligosaccharides. Preferably, the encapsulated soy extract is in spray dried form. The spray dried encapsulated soy extract may be used as a nutritional supplement or as an ingredient in a food item, beverage, cosmetic composition or pharmaceutical composition, to name a few of the uses of the invention. In particular the encapsulated soy extract may be a component of soft drinks, teas, juices, beverages, coffee, seasonings, confections, baked goods, alcoholic beverages, gum, chocolate, health food, dairy products, sauce, cereal, cosmetics and the like.

Oligosaccharides are carbohydrates are made up of monosaccharide units, which may be identical or different. Various oligosaccharides may be used in the invention. Preferably, however, the oligosaccharide is one or more cyclic oligosaccharide, for example cyclodextrin, such as a cyclodextrin selected from the group consisting of α-cyclodextrin, β-cyclodextrin, γ-cyclodextrin and combinations thereof.

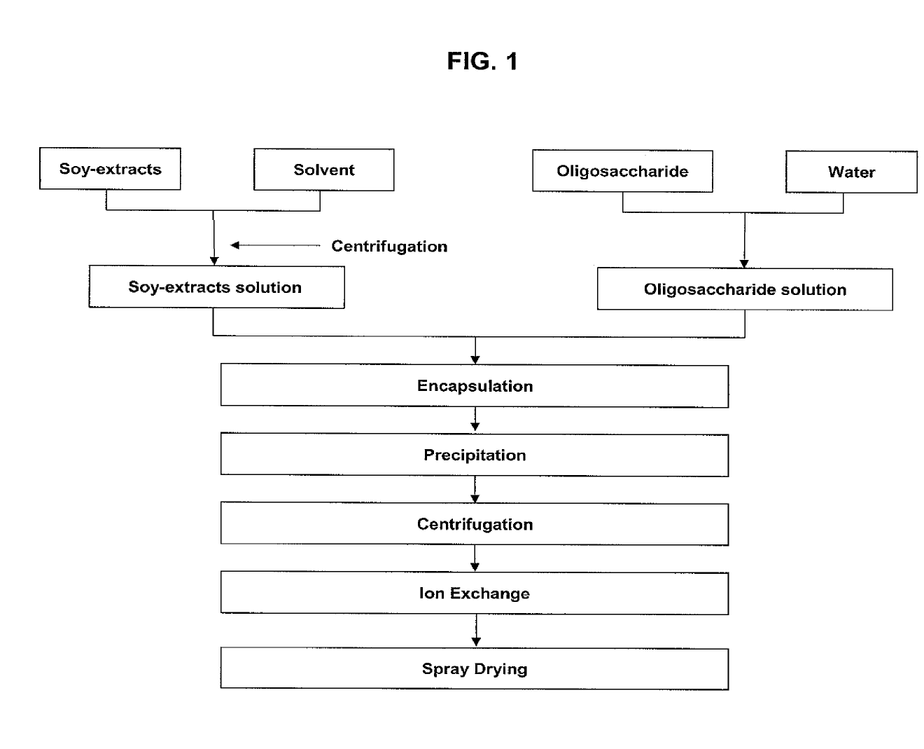

The processes for making the encapsulated soy extracts generally comprise the steps of providing a soy extract solution comprising one or more soy extracts and soy extract solvent, providing an oligosaccharide solution comprising one or more oligosaccharides and oligosaccharide solvent, combining the soy extract solution and oligosaccharide solution to encapsulate the soy extract with the oligosaccharide, precipitation of the encapsulated soy extract solution to form sediment comprising impurities, centrifugation, ion exchange to remove additional impurities from the separated soy extract and drying. The process results in soy extracts encapsulated with oligosaccharide, such as isoflavone derivatives encapsulated with β-cylcodextrin having little or no impurities and complete solubility in water. A general process for making the encapsulated soy extracts in accordance with an embodiment of the invention is set forth in FIG. 1.

In a preferred process for making the encapsulated soy extracts, a soy extract solution is formed by combining one or more soy extracts, such as, isoflavone or isoflavone derivative, with soy extract solvent, such as an alcohol. Soy extract solvents useful in the invention include those selected from the group consisting of water, ethanol, methyl alcohol, acetone, ether, isopropyl alcohol, hexane, carbon dioxide and combinations thereof. Ethanol and solutions of ethanol in water are preferred. The soy extracts and soy extract solvent may be mixed at ambient temperature for a sufficient time to thoroughly mix the soy extracts and soy extract solvent. Preferably, however, the soy extracts and soy extract solvent are mixed at a temperature of from about 30° C. to about 105° C., such as at about 50° C. to about 90° C. The soy extract and soy extract solvent may be mixed in a conventional mixer or stirrer. The soy extract solution comprises insoluble materials, which comprise impurities, including primary impurities. Some of the impurities, including primary impurities, i.e. fat and protein, however, enter solution with soy extract material. The insoluble materials are separated from the soy extract solution by a means for separation, which separates the insoluble materials from the solution. Examples of such means are centrifuging and filtration. The soy extract solution may be centrifuged using conventional centrifuging equipment, in a soy extract centrifuge step, to separate and remove insoluble materials from the soy extract solution. In an embodiment of the invention, the soy extract solution is centrifuged at about 500 rpm to about 22,500 rpm, such as at about 2,000 rpm to about 18,000 rpm.

The oligosaccharide solution is also formed for the process, and is preferably prepared separately from the preparation of the soy extract solution. One or more oligosaccharides, such as a cyclodextrin selected from the group consisting of α-cyclodextrin, β-cyclodextrin, γ-cyclodextrin and combinations thereof, are mixed with oligosaccharide solvent, such as water, optionally in the presence of an alkali, such as an alkali selected from the group consisting of potassium hydroxide, sodium hydroxide, magnesium hydroxide, calcium oxide, zinc oxide, aluminum oxide, sodium dicarbonate, magnesium oxide and combinations thereof. The oligosaccharide, solvent and optional alkali may be mixed in a conventional mixer or stirrer. In an embodiment of the invention, the oligosaccharide solution is prepared by combining and mixing oligosaccharide and optional alkali in solvent at a temperature so that the solids are dissolved thoroughly in the solvent, such as at about 30° C. to about 115° C., like at about 50° C. to about 100° C.

The soy extract solution and oligosaccharide solution are then combined in a means for encapsulation so that the soy extract, such as the isoflavone or isoflavone derivative, becomes encapsulated with the oligosaccharide. In an embodiment of the invention the encapsulation is conducted under agitation in a stirrer, such as a vessel comprising a magnetic stirrer, agitator, mixer, propeller, impeller or other mixing device. The means for encapsulation may also be a homomixer or homogenizer. The soy extract solution and oligosaccharide solution may be combined at a temperature of from about 40° C. to about 130° C., such as at about 50° C. to about 105° C., at about 100 revolutions per minute (“rpm”) to about 25,000 rpm, such as about 1,000 rpm to about 10,000 rpm, at about pH 4 to about pH 12, such as about pH 6 to about pH 10 for a period of time, such as for about 1 minute to about 10 hours, like about 10 minutes to about 3 hours. This step in the process results in an encapsulated soy extract mixture that comprises encapsulated soy extract and impurities, including primary impurities, i.e. protein and fat and the like, which eluted from soy extracts into the soy extract solution during the preparation of the soy extract solution.

After the encapsulation is complete, acid is optionally added to the encapsulated soy mixture to adjust the pH of the mixture and impurities, including primary impurities, are precipitated from the encapsulated soy extract mixture. In an embodiment of the invention, the pH may be adjusted to about 5 or less. An inorganic acid, organic acid or combinations thereof may be used. Acids selected from the group consisting of hydrochloric acid, phosphoric acid, sulfuric acid, nitric acid, carbonic acid, acetic acid, ascorbic acid, citric acid, fumaric acid, lactic acid, malic acid, tartaric acid and combinations thereof may be used. For example, hydrochloric acid may be added to the encapsulated soy extract mixture. The pH adjustment forms sediment of impurities, including primary impurities, from the encapsulated soy extract mixture obtained from the encapsulation step in the means for encapsulation. The sediment may then be separated and removed by centrifuging or filtering the encapsulated soy extract mixture. For example, the precipitated impurities may be removed by centrifuging the encapsulated soy extract mixture at about 500 rpm to about 25,000 rpm, such as at about 1,000 rpm to about 17,500 rpm. After centrifuging, an upper solution comprising the encapsulated soy extract mixture is formed and a pellet, at the bottom, comprising impurities, including primary impurities, is formed as sediment and removed.

During processing, particularly during the pH adjustment of the encapsulated soy extract mixture and separation of the precipitated impurities, ionic components are generated that can affect the taste of the final product. These ionic components are removed from the encapsulated soy extract mixture though an ion exchange.

After the sediment is removed from the encapsulated soy extract mixture, alkali is added to the encapsulated soy extract mixture to increase the pH and then ion exchange is conducted to remove ionic components from the encapsulated soy extract mixture. In an embodiment of the invention, alkali is added to increase the pH above about 3, such as to a pH of about 3.1 to about 9, like about 3.5 and then the ion exchange is conducted at a temperature of about 30° C. to about 110° C., such as at about 40° C. to about 90° C. The alkali added to increase the pH for the ion exchange may be selected from the group consisting of potassium hydroxide, sodium hydroxide, magnesium hydroxide, calcium oxide, zinc oxide, aluminum oxide, sodium dicarbonate, magnesium oxide and combinations thereof. The ion exchange may be conducted using an ion exchange resin or other conventional substrate.

In an embodiment, cationic exchange resins, anionic exchange resins or combinations thereof are used for the ion exchange resins. The used volume of cationic exchange resin may be about 0.1% to 100%, such as about 1% to about 5%, based on the volume of the encapsulated soy extract mixture. The used volume of anionic exchange resin may be about 0.1% to 100%, such as about 2% to about 10%, based on the volume of the encapsulated soy extract mixture.

Ion exchange may be performed by flowing encapsulated soy extract mixture through an ion exchange column filled with cationic exchange resin, anionic exchange resin or combinations thereof. Generally, the flow rate of the encapsulated soy extract mixture in the ion exchange column is about 0.1 ml/min to about 1000 l/min, such as at about 10 l/min to about 50 l/min.

In a further embodiment, the ion exchange resins, i.e. cationic exchange resins, anionic exchange resins or combinations thereof, can be added and stirred into a tank (or reservoir) of the encapsulated soy extract mixture in order to remove the ionic compounds. If adding the ion exchange resins into the tank, the stirred time may be about 1 minute to about 10 hours, such as at about 20 minutes to 90 minutes. The resin with the ionic components may then be separated from the encapsulated soy extract mixture, such as by a further centrifuging or filtering.

After the ion exchange, the encapsulated soy extract mixture comprises encapsulated soy extracts in solution with little or no impurities or primary impurities. The encapsulated soy extract mixture may then be dried such as by spray drying or freeze drying, to remove the solvent to obtain encapsulated soy extracts, such as encapsulated isoflavone derivatives. For example, the encapsulated soy extract mixture may be spray dried under any condition which results in the removal of the solvent to obtain a spray dried particulate material. In an embodiment of the invention, the encapsulated soy extract mixture is maintained at about the temperature of the ion exchange and processed through a spray drier having an inlet temperature of about 105° C. to about 250° C., such as at about 160° C. and an outlet temperature of about 60° C. to about 110° C., such as at about 85° C.

The invention pertains to a composition of matter comprising one or more soy extracts encapsulated with one or more oligosaccharides, including a composition of matter comprising isoflavone encapsulated with cyclodextrin wherein the encapsulated isoflavone comprises, little or no primary impurities and has complete or near complete water solubility. For example, isoflavone derivatives encapsulated with β-cyclodextrin having little or no primary impurities and complete or near complete water solubility. In an embodiment the encapsulated soy extracts, including encapsulated soy extracts made by the processes described herein, comprises oligosaccharide, such as cyclodextrin, i.e. β-cyclodextrin, isoflavone and less than about 1%, preferably less than about 0.7%, primary impurities, like about 0.1% to about 1.0% primary impurities, such as from about 0.1% to about 0.7% for example about 0.1% to about 0.9% protein and about 0.1% to about 0.9% fat. The encapsulated soy extracts have solubility in water, of about 99% to 100% based on a solution of about 1%.

The invention also concerns a process for encapsulating one or more soy extracts with one or more oligosaccharides comprising the steps of forming a soy extract solution and an oligosaccharide solution and combining the soy extract solution and oligosaccharide solution in a means for encapsulation to encapsulate the soy extracts with the oligosaccharides. The process may comprise the additional steps removing sediment, removing ionic components and drying. An aspect of the invention concerns a process for encapsulating isoflavone with cyclodextrin comprising the steps of forming a soy extract solution and a cyclodextrin solution and combining the soy extract solution and cyclodextrin solution in an encapsulation means to encapsulate the isoflavone of the soy extracts into the cyclodextrin. In any embodiment, the invention encompasses a process wherein the encapsulation means is a stirrer.

### Example

Spray dried isoflavone encapsulated with cyclodextrin was prepared. Initially, a cyclodextrin solution and soy extract solution were made. The cyclodextrin solution was obtained by placing 100 g of water into a vessel and then adding 10 g cyclodextrin and 150 mg NaOH and heating the vessel to 60° C. to dissolve the cyclodextrin and NaOH in the water. The soy extract solution was prepared by forming a 10 g ethyl alcohol solution by combining 5 g of ethanol (95% solution) and 5 g water in a vessel then heating to 50° C., adding 1 g of soy extracts, having a protein content of 8.9% dry weight basis and a fat content of 3.1% dry weight basis, mixing thoroughly under heat at 50° C. for 5 minutes and then centrifuging the mixture at 12,000 rpm to remove insoluble ingredients and obtain the soy extract solution.

The cyclodextrin solution and soy extract solution were then mixed in a stirrer to encapsulate soy extracts with the cyclodextrin. This step occurred in a stirrer at 50° C. and 500 rpm for 2 hours at a pH of about 5.5 to about 9.5. After encapsulation, about 1 ml of a 35% solution of HCl was added to the encapsulated soy extract mixture to generate sediment of impurities by adjusting pH to 4.5 or less and the sediment was removed from the encapsulated soy extract mixture by centrifugation at 2,000 rpm. Next, 5 mg NaOH was added to the encapsulated soy extract mixture to increase the pH above 4 and ionic components were removed by using an ion exchange process at 40° C. to 75° C.

After the ion exchange, the encapsulated soy extract mixture comprises a solution of isoflavone, i.e., isoflavone derivatives, encapsulated with cyclodextrin which is then spray dried to form particulate material of isoflavone, i.e., isoflavone derivatives, encapsulated with cyclodextrin. The solution of isoflavone encapsulated with cyclodextrin is maintained at 50° C. and dried in a spray drier having an inlet temperature of 160° C. and an outlet temperature of 85° C. Analysis of the final product indicated a protein content of 0.6% dry weight basis and a fat content of 0.1% dry weight basis.

1. A process for making encapsulated soy extracts comprising the steps of:
a) providing a soy extract solution comprising one or more soy extracts and a soy extract solvent;
b) providing an oligosaccharide solution comprising one or more oligosaccharides and oligosaccharide solvent;
c) combining the soy extract solution and oligosaccharide solution to form an encapsulated soy extract mixture;
d) precipitating impurities from the encapsulated soy extract mixture;
e) separating the precipitated impurities from the encapsulated soy extract mixture;
f) adding alkali to the encapsulated soy extract mixture to increase the pH and then conducting ion exchange to remove ionic components from the encapsulated soy extract mixture; and
g) drying the encapsulated soy extract mixture.

2. The process of claim 1 wherein the soy extract solution is obtained by mixing one or more soy extracts with soy extract solvent at a temperature of about 30° C. to about 105° C. and then centrifuging the mixed soy extracts and soy extract solvent at about 500 rpm to about 22,500 rpm.

3. The process of claim 2 wherein the soy extract solvent is selected from the group consisting of water, ethanol, methyl alcohol, acetone, ether, isopropyl alcohol, hexane, carbon dioxide and combinations thereof.

4. The process of claim 1 wherein the oligosaccharide solution is obtained by mixing one or more oligosaccharides with water at about 30° C. to about 115° C.

5. The process of claim 4 wherein alkali is added to the oligosaccharide solution.

6. The process of claim 5 wherein the alkali added to the oligosaccharide solution is selected from the group consisting of potassium hydroxide, sodium hydroxide, magnesium hydroxide, calcium oxide, zinc oxide, aluminum oxide, sodium dicarbonate, magnesium oxide, and combinations thereof.

7. The process of claim 1 wherein the soy extract is an isoflavone derivative.

8. The process of claim 7 wherein the isoflavone derivative is selected from the group consisting of daidzin, glycitin, genistin, daidzein, glycitein, genistein and combinations thereof.

9. The process of claim 1 wherein the oligosaccharide is selected from the group consisting of α-cyclodextrin, β-cyclodextrin, γ-cyclodextrin and combinations thereof.

10. The process of claim 1 wherein the soy extract solution and oligosaccharide solution are combined by mixing at about 40° C. to about 130° C. at about 100 rpm to about 25,000 rpm and at about pH 4 to about pH 12.

11. (canceled)

12. The process of claim 10 wherein the soy extract solution and oligosaccharide solution are combined for a period of time of about 1 minute to about 10 hours.

13-17. (canceled)

18. The process of claim 1 wherein the encapsulated soy extract mixture is centrifuged at about 500 rpm to about 25,000 rpm.

19-22. (canceled)

23. The process of claim 1 wherein the ion exchange is performed by flowing the encapsulated soy extract mixture through an ion exchange column filled with exchange resin selected from the group consisting of cationic exchange resin, anionic exchange resin and combinations thereof.

24-25. (canceled)

26. The process of claim 1 wherein the ion exchange comprises adding ion exchange resins to the encapsulated soy extract mixture, stirring the encapsulated soy extract mixture and ion exchange resins for about 1 minute to about 10 hours to form ionic components and separating the ionic components from the encapsulated soy extract mixture.

27-30. (canceled)

31. An encapsulated soy extract made by the process of claim 1.

32. An encapsulated material comprising one or more soy extracts encapsulated with oligosaccharide wherein the encapsulated material comprises less than about 1% primary impurities and has solubility in water of about 99% to 100% based on a solution of about 1%.

33. The encapsulated material of claim 32 wherein the soy extract is an isoflavone derivative.

34. The encapsulated material of claim 33 wherein the isoflavone derivative is selected from the group consisting of daidzin, glycitin, genistin, daidzein, glycitein, genistein and combinations thereof.

35. The encapsulated material of claim 32 wherein the oligosaccharide is selected from the group consisting of σ-cyclodextrin, β-cyclodextrin, γ-cyclodextrin and combinations thereof.

36. The encapsulated material of claim 32 comprising less than about 0.7% primary impurities.

37-38. (canceled)
